(19) United States
(12) Patent Application Publication
(10) Pub. No.: US 2023/0236169 A1
(43) Pub. Date: Jul. 27, 2023

(54) NANOPLASMONIC BIOSENSOR FOR DETECTING AUTOPHAGY MARKER WITH HIGH SENSITIVITY, AND METHOD OF DETECTING AUTOPHAGY MARKER AND METHOD OF SCREENING DRUG CANDIDATE USING SAME

(71) Applicant: KOREA UNIVERSITY RESEARCH AND BUSINESS FOUNDATION, Seoul (KR)

(72) Inventors: Sang Jun SIM, Seoul (KR); Jong Uk LEE, Seoul (KR); Young Jae CHOI, Seoul (KR)

(73) Assignee: KOREA UNIVERSITY RESEARCH AND BUSINESS FOUNDATION, Seoul (KR)

(21) Appl. No.: 18/095,107

(22) Filed: Jan. 10, 2023

(30) Foreign Application Priority Data
Nov. 10, 2021 (KR) 10-2021-0154028
Nov. 10, 2022 (KR) 10-2022-0149414

(51) Int. Cl. G01N 33/50; G01N 33/68

(52) U.S. Cl. CPC G01N 33/5011; G01N 33/6875

(57) ABSTRACT

Disclosed are a nanoplasmonic biosensor for detecting an autophagy marker with high sensitivity using a plasmon resonance effect, and a method of detecting an autophagy marker and a method of screening a cancer therapeutic agent using the same.

### CROSS-REFERENCE TO RELATED APPLICATIONS

This application claims the benefit under 35 USC § 119(a) of Korean Patent Application No. 10-2021-0154028 filed on Nov. 10, 2021 and Korean Patent Application No. 10-2022-0149414 filed on Nov. 10, 2022 in the Korean Intellectual Property Office, the entire disclosures of which are incorporated herein by reference for all purposes.

### BACKGROUND OF THE INVENTION

### Field of the Invention

The present invention relates to a nanoplasmonic biosensor for detecting an autophagy marker with high sensitivity using a plasmon resonance effect, and a method of detecting an autophagy marker and a method of screening a cancer therapeutic agent using the same.

### Description of the Related Art

Autophagy is a biological phenomenon that occurs within eukaryotic cells and also that maintains cell homeostasis through destruction and recycling of cell components, and is a term referring to the overall process in which intracellular components are assembled in a double membrane called an autophagosome and degraded by lysosomes.

LC3 is a protein mainly used for measuring autophagy, and LC3-I is processed into LC3-II during autophagy to form autophagosomes, and as such, a detailed concentration of LC3 is known to be closely related to the degree of activation of autophagy. It has also been clinically proven that the concentration of LC3 may be used as a significant indicator not only for measuring the degree of activation of autophagy, but also for cancer diagnosis and prognosis after drug treatment. Moreover, reduction in effectiveness of anticancer drugs due to autophagy mechanisms has also been proven through a number of studies.

In particular, it has recently been known that the degree of activation of autophagy is closely related to various diseases such as brain disease, obesity, aging, cancer, and the like and thus research into measurement of autophagy is becoming more important.

In addition, since new anticancer drugs targeting autophagy are being actively developed for clinical trials, accurate autophagy (flux) assay is required, and since patients prefer minimal tumor resection, the LC3 turnover-autophagic flux assay requires high sensitivity. However, anti-LC3 antibodies recognize both LC3-I and LC3-II, making it impossible to quantify individual LC3 forms based on current LC3 assays.

Conventionally, LC3 Western blotting, which produces two bands (LC3-I/II) near 15 kDa, is one of the most favored and routinely used techniques for analyzing autophagic flux, but merely provides relative quantification between samples loaded on a single gel, and the procedure is complex and technically demanding. LC3 ELISA shows a broad dynamic range of detection with higher sensitivity, but is limited to total LC3 quantification alone. Compared to Western blot results, LC3 ELISA inevitably has disadvantages in that it lacks accurate quantification and is laborious, expensive, and complicated to estimate LC3-I and LC3-II concentrations for LC3 turnover assay. Therefore, simple and accurate methods capable of quantifying individual LC3 forms are still needed to develop rapid drug screening tools for autophagy-targeting cancer therapies.

Furthermore, for patient samples obtained in the clinical process, the concentration of cells is much lower than that of cell lines artificially cultured in laboratories, and the amount of protein that is expressed is also very small, and thus a new, fast, and inexpensive test method is still needed in order to be used in actual medical practice.

Recently, as an alternative thereto, a plasmonic biosensor platform capable of real-time and label-free detection with high selectivity and sensitivity is receiving attention. In nanometer-scale metal nanostructures such as nanoparticles and nanorods constituting a plasmonic biosensor, free electrons surrounding the nanostructures oscillate collectively by light in a specific wavelength range incident from the outside, thus showing electric dipole characteristics, thereby strongly scattering and absorbing light in the corresponding frequency range, which is called localized surface plasmon resonance (LSPR). The scattering and absorption of LSPR have characteristics of responding sensitively depending on the shape and size of the metal nanostructure and the dielectric environment surrounding the nanostructure. For this reason, nanoplasmonic biosensors have the potential to be utilized as a platform capable of detecting biological molecules and biochemical bonds that occur on the surface of nanoparticles depending on changes in localized refractive index.

Against this background, the present inventors have ascertained that a localized surface plasmon resonance (LSPR)-based nanoplasmonic sensor may detect LC3 as an autophagy marker with low sensitivity and in a wide concentration range, even without an additional marker, and may determine individual LC3-I and LC3-II compositions, making it possible to quantify concentrations thereof based thereon, thereby culminating in the present invention.

### SUMMARY OF THE INVENTION

An object of the present invention is to provide a method of detecting an autophagy marker with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection.

Another object of the present invention is to provide a method of determining autophagic flux with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection.

Still another object of the present invention is to provide a method of screening a cancer-targeting drug candidate with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods.

In order to accomplish the above objects, the present invention provides a method of detecting an autophagy marker with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection

In an embodiment of the present invention, the immunogold nanorods may have an aspect ratio of 3 to 4.

In another embodiment of the present invention, the biosensor may detect the autophagy marker by measuring a change in Rayleigh scattering spectrum generated by the specific binding of the autophagy marker.

In still another embodiment of the present invention, the autophagy marker may be LC3.

In yet another embodiment of the present invention, the LC3 may include LC3-I and LC3-II.

In still yet another embodiment of the present invention, the monoclonal antibody may be LC3-mAb.

In even yet another embodiment of the present invention, the biosensor may detect the autophagy marker in a range of femtomolar concentration (fM) to nanomolar concentration (nM).

In a further embodiment of the present invention, the biosensor may detect the autophagy marker at a low limit of detection ranging from 10² fM to 10⁶ fM.

In still a further embodiment of the present invention, the method may further include treating the surface of the substrate of the biosensor, on which the immunogold nanorods are immobilized, with carboxymethyl-polyethylene glycol-thiol.

In yet a further embodiment of the present invention, the biomarker mixture may be a cancer-cell-derived lysate.

In addition, the present invention provides a method of determining autophagic flux with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection.

In an embodiment of the present invention, the method may quantify a total concentration of LC3 in a sample in which LC3-I and LC3-II are mixed at various concentrations, and simultaneously may quantify a ratio of LC3-I to LC3-II.

In another embodiment of the present invention, the method may further include (3) measuring a ratio of LC3-I to LC3-II by substituting the two maximum wavelength shift values obtained in steps (1) and (2) into Equation 1 below.

Z=1−2.072×(Y−0.3265)/(X−0.3974) [Equation 1]

Here, X is the first LSPR peak shift value, Y is the second LSPR peak shift value, and Z is the LC3-II ratio.

In Equation 1, R²=0.9805, in which the R-squared value is “the ratio of the difference between the target variance and the variance of the prediction error for the target variance” upon fitting using a program based on experimentally obtained data, and is a numerical value that shows how well the data used to build the model matches the fit. The closer R² is to 1, the more accurate it is, and if it exceeds 0.95, it may be interpreted as a very reliable fit.

In still another embodiment of the present invention, the autophagic flux may be analyzed by quantifying LC3-I and LC3-II within a concentration range of 10² to 10⁶ fM.

In addition, the present invention provides a method of screening a cancer-targeting drug candidate with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) treating a cancer cell line with an autophagy inhibitor, (2) measuring a first maximum wavelength shift value by bringing a cell lysate isolated from the cancer cell line after step (1) into contact with immunogold nanorods, and measuring a second maximum wavelength shift value after PEBP1 injection, (3) treating the cancer cell line with a cancer-targeting drug candidate, (4) measuring a first maximum wavelength shift value by bringing a cell lysate isolated from the cancer cell line after step (3) into contact with immunogold nanorods, and measuring a second maximum wavelength shift value after PEBP1 injection, (5) calculating a composition of LC3-I and LC3-II by substituting the first and second maximum wavelength shift values obtained in steps (2) and (4) into Equation 1 below, and (6) determining the drug candidate to be a cancer therapeutic agent when LC3 and LC3-II values calculated in step (4) are increased compared to LC3 and LC3-II values calculated in step (2):

Z=1−2.072×(Y−0.3265)/(X−0.3974) [Equation 1]

in which X is the first LSPR peak shift value, Y is the second LSPR peak shift value, Z is the LC3-II ratio, and R²=0.9805.

In an embodiment of the present invention, the autophagy inhibitor may include bafilomycin A, chloroquine, 3-methyladenine, or combinations thereof.

In another embodiment of the present invention, the cell lysate may include LC3-I and LC3-II that are mixed at various concentrations.

### BRIEF DESCRIPTION OF THE DRAWINGS

FIG. 1 schematically shows a closed water-bath sensing chamber and a microfluidic channel therein;

FIG. 2 schematically shows a biosensor and an autophagy marker detection process according to the present invention;

FIG. 3 shows a normalized UV-vis spectrum of immunogold nanorods (AuNRs);

FIGS. 4A and 4B show dark-field images of immunogold nanoparticles immobilized on an APTES-treated glass slide;

FIG. 5 shows a typical Rayleigh scattering spectrum of individual gold nanorods according to the Lorentzian algorithm, in which the LSPR wavelength peaks are red-shifted for two sensing steps and the inset box shows the corresponding molecular binding of LC3 and PEBP1;

FIGS. 6A to 6D show selectivity and sensitivity of LC3 sensing a two-step LSPR sensing platform, error bars being standard errors measured in 60 nanorods;

FIGS. 7A to 7C show the LSPR peak shift range and linearity in the first and second LSPR sensing steps, and the confidence interval calculated using software;

FIG. 8 shows the optimal concentration of PEBP1 injected in the second LSPR sensing step;

FIGS. 9A to 9C show the absence of LC3-I after treatment with cytosolic LC3-I protease by Western blotting and ELISA;

FIG. 10 shows the first step in various LC3 samples having the same total LC3 concentration but different LC3-I/II ratios;

FIG. 11A shows 3D bar graphs of additional LSPR red-shifts in the second LSPR sensing step for cell lysate samples having different LC3-I and LC3-II compositions, and FIG. 11B shows 25 experimental results for five different LC3 concentrations and compositions, in which the first LSPR peak shift, the second LSPR peak shift, and the LC3-II ratio are represented in 3D on the xyz axes;

FIG. 12 shows data within a concentration range of 10² to 10⁶ fM as represented by 3D surface equation 1 using OriginPro 9.0 software (Equation 1), in which the xyz axes represent the first and second LSPR peak shifts and the LC3-II ratio;

FIG. 13 is a cumulative bar graph showing LSPR peak shifts of a Caov-3 cell line treated with bafilomycin A or not treated therewith;

FIG. 14 shows results of LC3 Western blotting and LC3 ELISA of cell lysates derived from Caov-3 cells treated with different concentrations of bafilomycin A (Baf A) for 20 minutes and 24 hours;

FIGS. 15A and 15B show LC3-II ratios calculated by substituting the LSPR peak shifts of cell lysates derived from two different human breast cancer cell lines under different conditions (20 μg mL⁻¹ trastuzumab and 50 nM bafilomycin A for 12 hours) into Equation 1;

FIGS. 16A and 16B show changes in cell viability over time after treatment with 20 μg mL⁻¹ trastuzumab and 50 nM bafilomycin A in the growth medium of BT-474 and MDA-MB-231 human breast cancer cells; and

FIGS. 17A and 17B show results of LC3 analysis of cell lysates treated with bafilomycin A and trastuzumab for 12 hours, quantified using Western blotting and LC3 ELISA kits of LC3 expression in BT-474 and MDA-MB-231 cell lines.

### DETAILED DESCRIPTION OF THE INVENTION

The present invention is described in detail. The description and embodiments disclosed in the present invention may be applied to other descriptions and embodiments. Specifically, all combinations of various elements disclosed herein fall within the scope of the present invention. Also, the following description is not to be construed as limiting the scope of the present invention.

Moreover, those skilled in the art will be able to recognize or ascertain many equivalents to specific embodiments of the invention described herein using no more than routine experimentation. Also, such equivalents are intended to be encompassed by the present invention.

The present invention has been made keeping in mind the problems in that Western blotting or ELISA has limitations in detecting autophagy markers and compositions.

Accordingly, an aspect of the present invention pertains to a method of detecting an autophagy marker with a nanoplasmonic biosensor for measuring an autophagy marker with high sensitivity using immunogold nanorods.

Specifically, the method of detecting an autophagy marker with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods includes (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection.

Hereinafter, a detailed description will be given of the present invention.

As used herein, the term “biosensor” is a device for sensing using a special reaction between a biological material such as an enzyme and an antibody and a molecule to be detected in a complex mixture, and the biosensor of the present invention may be used to detect an autophagy marker.

As used herein, the term “autophagy” is a biological phenomenon that occurs within eukaryotic cells and also that maintains cell homeostasis through destruction and recycling of cellular components, and is a term referring to the overall process in which intracellular components are assembled in a double membrane called an autophagosome and degraded by lysosomes.

Among autophagy markers, LC3 is a protein mainly used to measure autophagy. During autophagy, since LC3-I is processed into LC3-II to form autophagosomes, the detailed concentration of LC3 is known to be closely related to the degree of activation of autophagy. It has been clinically proven that the concentration of LC3 may be used as a significant indicator not only for measuring the degree of activation of autophagy, but also for cancer diagnosis and prognosis after drug treatment. Moreover, reduction in effectiveness of anticancer drugs due to autophagy mechanisms has also been proven through many studies.

As used herein, the term “nanoplasmonic biosensor” is a biosensor capable of measuring a plasmon, in which a plasmon is a quantum of electron or hole density oscillation, namely a quantum of plasma oscillation, and refers to a biosensor including a measurement unit capable of measuring a plasmon, which is a quasiparticle in which free electrons in the metal oscillate collectively.

For example, the biosensor is capable of measuring a change in the Rayleigh scattering spectrum of immunogold nanoparticles generated through specific binding of a mixture containing an autophagy marker to a monoclonal antibody specifically binding to the autophagy marker.

The biosensor is capable of detecting an autophagy marker by measuring a change in Rayleigh scattering spectrum caused by specific binding of the autophagy marker.

Also, the biosensor is capable of detecting an autophagy marker in a wide range of femtomolar concentration to nanomolar concentration.

According to an embodiment of the present invention, the biosensor is capable of detecting an autophagy marker even at a low limit of detection. Here, the low limit of detection represents a range of 10¹ fM to 10⁸ fM, particularly 10² fM to 10⁶ fM, without being limited thereto.

In the present invention, “immunogold nanorods” refer to particles capable of measuring the region and quantity of an antigen in cells or tissues by labeling immunogold, and may be used interchangeably with immunogold, immunogold nanoparticles, or alternatively gold nanorods or gold nanoparticles excluding “immuno”.

The immunogold nanorods may have an aspect ratio of 2 to 5, particularly 3 to 4, without being limited thereto.

The immunogold nanorods may be immunogold nanoparticles linked with a monoclonal antibody specifically binding to an autophagy marker.

The term “monoclonal antibody” refers to an antibody that reacts with only one antigenic determinant, particularly a monoclonal antibody that specifically binds only to LC3 protein.

The monoclonal antibody linked to the immunogold nanorods may specifically bind to an autophagy marker to thus generate a change in Rayleigh scattering spectrum, and as can be seen from results of the following examples, it may be particularly LC3-mAb.

The term “substrate” refers to a plate on which the immunogold nanoparticles may be immobilized so that they are observed under a microscope, and particularly a glass slide, but is not limited thereto.

The term “measurement unit” refers to a unit configured to measure localized surface plasmon resonance in the immunogold nanorods, and is not limited, so long as it is able to measure localized surface plasmon generated when the LC3 protein binds to an antibody that specifically binds to LC3 conjugated to the immunogold nanorods.

As confirmed in a specific embodiment of the present invention, when the biosensor is configured to include immunogold nanorods, which are manufactured, conjugated with a monoclonal antibody specific to LC3 protein, and immobilized on a glass slide and a measurement unit capable of measuring localized surface plasmon resonance caused by binding of LC3 protein to an antibody that specifically binds to LC3 conjugated to the immunogold nanorods, the biosensor may effectively detect LC3 at a low limit of detection and in a wide detection range compared to conventionally known ELISA or Western blotting.

The method of detecting autophagy may largely include steps (1) and (2).

Step (1) is measuring a Rayleigh scattering spectrum and obtaining a maximum wavelength shift therefrom by bringing a biomarker mixture into contact with the immunogold nanorods to induce specific binding to the monoclonal antibody. Here, the terms “immunogold nanorods” and “monoclonal antibody” are as described above.

The term “biomarker mixture” refers to a mixture in which a biomarker to be detected in the present invention is mixed, particularly a cell lysate, more particularly a cancer-cell-derived cell lysate. Any mixture including an autophagy-marker-related biomarker to be measured in the present invention may be included without limitation.

Next, step (2) is measuring a Rayleigh scattering spectrum and obtaining a maximum wavelength shift therefrom by inducing specific binding to LC3-I after PEBP1 injection.

The term “PEBP1 (phosphatidylethanolamine binding protein 1)” refers to a protein containing the LIR (LC3 interacting region) motif of WDGL (W: tryptophan, D: aspartic acid, G: glycine, L: leucine) (55-58 amino acids of human PEBP1), which binds to LC3-I but not to LC3-II. In this respect, the LC3 nanoplasmonic biosensor of the present invention suggests that the total amount of LC3 and the specific composition of LC3-I and LC3-II may be analyzed within a single platform.

The method of detecting the autophagy marker according to the present invention enables label-free detection, that is, detection without a label, and may further include, before step (1), treating the substrate of the nanoplasmonic biosensor, on which the immunogold nanoparticles are immobilized, with carboxymethyl-polyethylene glycol-thiol.

The biosensor of the present invention may be manufactured by (a) synthesizing gold nanorods by adding a growth solution to seeds in a mixture of CTAB and sodium oleate, (b) replacing CTAB on the surface of the gold nanorods with carboxymethyl-polyethylene glycol-thiol (CM-PEG-SH), (c) synthesizing monoclonal-antibody-linked immunogold nanorods by adding the gold nanorods of step (b) to a monoclonal antibody specifically binding to an autophagy marker, and (d) immobilizing the monoclonal-antibody-linked immunogold nanoparticles on a substrate.

As used herein, the terms “autophagy”, “nanoplasmonic biosensor”, “immunogold nanorods”, “monoclonal antibody”, and “substrate” are as described above.

In step (b) of replacing CTAB on the surface of the gold nanorods with carboxymethyl-polyethylene glycol-thiol (CM-PEG-SH), carboxymethyl-polyethylene glycol-thiol is used to optimize binding to the surface of the gold nanorods and binding to the monoclonal antibody. This is because the terminal of carboxymethyl-polyethylene glycol-thiol contains —SH groups used for binding to the surface of gold nanoparticles and —NH groups used for binding to the monoclonal antibody.

In step (c), the monoclonal antibody is linked to the surface of the gold nanorods to form specific binding to the biomarker. Here, any monoclonal antibody may be used, so long as it is capable of generating a change in Rayleigh scattering spectrum by specifically binding to a biomarker, but as can be seen from results of the following examples, it may be LC3-mAb.

Also, before step (b), in order to increase efficiency of binding to the gold nanoparticles, the monoclonal antibody may be mixed with a mixed solution of 1-ethyl-3-(3-dimethylaminopropyl)carbodiimide (EDC) and N-hydroxysuccinimide (NHS) such that the carboxyl group of the antibody is converted into an NHS ester.

Another aspect of the present invention pertains to a method of determining autophagic flux with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods, the method including (1) measuring a Rayleigh scattering spectrum and obtaining a maximum wavelength shift therefrom by bringing a biomarker mixture into contact with immunogold nanorods to induce specific binding to a monoclonal antibody and (2) measuring a Rayleigh scattering spectrum and obtaining a maximum wavelength shift therefrom by inducing specific binding to LC3-I after PEBP1 injection.

In the present invention, the terms “autophagy”, “nanoplasmonic biosensor”, “immunogold nanorods”, “monoclonal antibody”, “substrate”, “measurement unit”, “autophagy marker”, “biomarker mixture”, and “PEBP1” are as described above.

The term “autophagic flux” refers to the degree to which autophagy occurs. As new anticancer drugs targeting autophagy are currently being actively developed for clinical trials, accurate autophagy (flux) assay is required, and also, since patients prefer minimal tumor resection, the LC3 turnover-autophagic flux assay requires high sensitivity to analyze the degree of autophagy or autophagic flux, thereby determining the degree to which autophagy has progressed.

The method is characterized in that the total concentration of LC3 is quantified in a sample in which LC3-I and LC3-II are mixed at various concentrations, and simultaneously the ratio of LC3-I to LC3-II is quantified.

The method may further include (3) measuring the ratio of LC3-I to LC3-II by substituting the two maximum wavelength shift values obtained in steps (1) and (2) into Equation 1 below:

Z=1−2.072×(Y−0.3265)/(X−0.3974) [Equation 1]

Here, X is the first LSPR peak shift value, Y is the second LSPR peak shift value, Z is the LC3-II ratio, and R²=0.9805.

The method may be characterized in that autophagic flux is analyzed by quantifying LC3-I and LC3-II within a concentration range of 10¹ to 10⁸ fM, particularly 10² to 10⁶ fM.

Still another aspect of the present invention pertains to a method of screening a cancer-targeting drug candidate with a nanoplasmonic biosensor for measuring an autophagy marker with high sensitivity.

Particularly, the method of screening a cancer-targeting drug candidate with a nanoplasmonic biosensor for measuring an autophagy marker with high sensitivity including a substrate, immunogold nanorods immobilized on the substrate and linked with a monoclonal antibody specifically binding to an autophagy marker, and a measurement unit configured to measure localized surface plasmon resonance in the immunogold nanorods may include (1) treating a cancer cell line with an autophagy inhibitor, (2) measuring a first maximum wavelength shift value by bringing a cell lysate isolated from the cancer cell line after step (1) into contact with immunogold nanorods, and measuring a second maximum wavelength shift value after PEBP1 injection, (3) treating the cancer cell line with a cancer-targeting drug candidate, (4) measuring a first maximum wavelength shift value by bringing a cell lysate isolated from the cancer cell line after step (3) into contact with immunogold nanorods, and measuring a second maximum wavelength shift value after PEBP1 injection, (5) calculating the composition of LC3-I and LC3-II by substituting the first and second maximum wavelength shift values obtained in steps (2) and (4) into Equation 1 below, and (6) determining the drug candidate to be a cancer therapeutic agent when the LC3 and LC3-II values calculated in step (4) are increased compared to the LC3 and LC3-II values calculated in step (2).

Z=1−2.072×(Y−0.3265)/(X−0.3974) [Equation 1]

Here, X is the first LSPR peak shift value, Y is the second LSPR peak shift value, Z is the LC3-II ratio, and R²=0.9805.

In the present invention, the terms “autophagy”, “nanoplasmonic biosensor”, “immunogold nanorods”, “monoclonal antibody”, “substrate”, “measurement unit”, “autophagy marker”, “cell lysate”, “PEBP1”, and “Equation 1” are as described above.

As used herein, the term “autophagy inhibitor” refers to a material that inhibits autophagy occurring in cells, particularly bafilomycin A, chloroquine, 3-methyladenine, or combinations thereof, but is not limited thereto.

In the method of screening the cancer-targeting drug, the cancer cell line is sequentially treated with an autophagy inhibitor and a drug candidate, and the total amount of LC3 and the composition (ratio) of LC3-I and LC3-II are measured based on LSPR maximum wavelength shifts, after which, when the LC3 and LC3-II values calculated after treatment with the drug candidate are increased compared to the LC3 and LC3-II values calculated after treatment with the autophagy inhibitor, the drug candidate is capable of inducing autophagy in cancer cells to treat cancer, and may thus be determined to be a cancer therapeutic agent.

Also, the term “cell lysate” is similar to the biomarker mixture described above, and may be a cell lysate derived from a cancer cell line, or a mixture of LC3-I and LC3-II at various concentrations.

In other screening methods, molecular biology assays, biochemical assays, etc. known in the art may be performed, and exemplarily, methods described in Examples of the present specification may be used.

In a specific embodiment of the present invention, the new plasmonic nanoparticle-based two-step LSPR sensing platform capable of accurate LC3 turnover calculation for autophagic flux assay may exhibit 20-fold higher sensitivity than conventional LC3 ELISA by quantifying total LC3 in the range of femtomolar concentration to nanomolar concentration, and may quantify individual LC3 forms by measuring the LC3 composition when compared to relative values obtained through Western blotting. In addition, experiments using samples mimicking clinical samples in which LC3-I and LC3-II are mixed show that the biosensor of the present invention provides reliable and accurate sensing results and a specific equation representing the correlation between LSPR shift and LC3 composition is obtained. In addition, whether an anticancer drug candidate is useful as an anticancer drug may be screened by classifying LC3 expression levels in drug-treated human cancer cell lines in detail and by measuring the degree of autophagy of autophagy-targeting drugs using the new nanoplasmonic biosensor.

A better understanding of the present invention may be obtained through the following examples. These examples are merely set forth to illustrate the present invention and are not to be construed as limiting the scope of the present invention.

### Experimental Example 1: Chemicals and Materials

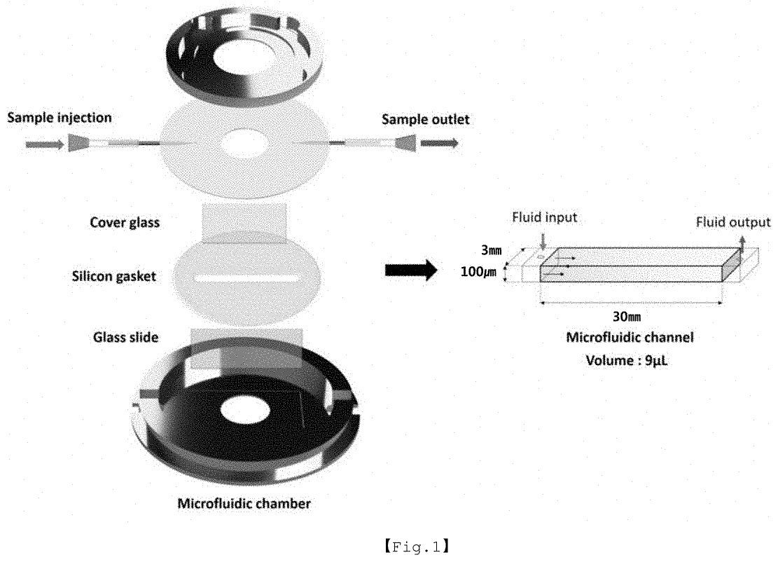

Sodium borohydride (NaBH₄, 99%), sodium oleate (NaOL, >97.0%), gold(III) chloride trihydrate (≥99.0%), hexadecyltrimethylammonium bromide (CTAB, >98.0%), N-hydroxysuccinimide (NHS), N-ethyl-N-(diethylaminopropyl)carbodiimide (EDC), hydrochloric acid (HCl, 37 wt % in water), 3-aminopropyltriethoxysilane (APTES, 99.0%), bafilomycin A, and LC3B monoclonal antibody were purchased from Sigma Aldrich (Korea). SH-PEG-COOH (Mw: 5,000) was purchased from Laysan Bio, Inc. LC3-I human recombinant protein was purchased from Enzo Life Science. PEBP1 (recombinant human RKIP protein) and ATG4B knockout cell lysates were purchased from Abcam. LC3 ELISA kits were purchased from Cell Signaling Technology and Cell Biolabs. FBS, RPMI-1640, antibiotic-antimycotic, and DMEM were purchased from Hyclone. Trastuzumab (anti-Her2) was purchased from Selleckchem (Korea). Ultrapure water (18.2 mΩ cm⁻¹) was prepared by high-pressure sterilization after deionization and purification with aquaMAX Ultra 370 series (Youngrin, Korea). A sensing chamber was separately designed so as to be suitable for an LSPR system and manufactured through CNC machining (FIG. 1).

### Experimental Example 2: Preparation of Cell Lysate Sample of Cultured Cells

Caov-3, BT-474, and MDA-MB-231 cell lines were purchased from the Korean Cell Line Bank (Seoul, Korea). Each cell line was cultured in a 10-cm dish under appropriate conditions before cell lysate preparation. The human ovarian cancer cell line Caov-3 was grown in DMEM (10% FBS), and the human breast cancer cell lines BT-474 and MDA-MB-231 were grown in RPMI-1640 (10% FBS, 1% antibiotic-antimycotic). Caov-3 cells were treated with bafilomycin A1 (1 μM) and allowed to starve for 24 to 48 hours before adding a cytosolic LC3 removal reagent. BT-474 and MDA-MB-231 were selectively treated with trastuzumab (20 μg mL⁻¹) and bafilomycin A1 (50 nM). Pellet cells were then washed with ice-cold PBS and incubated for 15 minutes with RIPA lysis buffer and inhibitors (Halt™ Protease and Phosphatase Inhibitor Cocktail). Thereafter, sonication and centrifugation were repeated to afford a cell lysate at a final concentration of 1.6×10⁶ cells/mL.

### Experimental Example 3: Western Blotting

The cell lysate prepared in Experimental Example 2 was quantified with a BCA assay kit (Thermo Fisher Scientific), and 40 μg thereof was loaded into each well. SDS-PAGE was conducted under conditions of 70 minutes or more, 15% gel, and 90 V. Primary (LC3B antibody, Cell Signaling) and secondary (goat anti-mouse, GenDEPOT) antibodies to LC3 were diluted 1:1000 and 1:5000, respectively, and respective antibodies to actin (anti beta-actin, AbFrontier) were also diluted 1:1000 and 1:10000. Other specific conditions were the same as the general protocol for Western blotting.

### Experimental Example 4: Cell Viability

Each cell line was preincubated in 96 wells at 5×10⁴ cells per well in a CO₂ incubator (37° C., 5% CO₂, 2-24 hours). Trastuzumab (20 μg/mL) and bafilomycin A1 (50 nM) were added thereto, followed by incubation for 6, 24, and 48 hours. Each well was treated with 10 μL of EZ-Cytox (DoGen Bio, Korea), followed by further incubation for at least 1 hour. Thereafter, UV-vis absorbance at 450 nm was recorded.

### Experimental Example 5: Method of Calculating Limit of Detection (LOD)

LOD was estimated without LC3 giving a 3-fold or higher LSPR signal than background. The standard curve of LC3 is represented as follows.

Y=A+B×X (1)

Here, A and B are the variables obtained by least squares linear regression on the signal concentration curve for variable Y representing the LSPR signal at the LC3 concentration of X(fM). For LC3-I herein, A1 is −1.0262 and B1 is 2.8114. For LC3-II, A2 is −1.0524 and B2 is 2.8925.

Y=Yblank+3SD (2)

Here, SD is the standard deviation and Yblank is the LSPR signal of blank samples (excluding LC3). For the sensor of the present invention, LC3 KO cell lysates were tested and respective values of 1.86 and 0.75 were obtained.

LOD was calculated as follows.

LOD=10(Yblank+3SD)−AB (3)

SD was calculated according to a well-known equation.

SD=√1N−1×Σ(Xi−Xaverage)2Ni=1 (4)

Here, N is the total number of LC3 standard samples. Xi is the “I” sample of the series of measured values. Xaverage is the average of the LSPR signals obtained for a specific series of identical samples repeated N times.

LODs were calculated to be 66.77 fM and 60.61 fM for LC3-I and LC3-II, respectively.

### Experimental Example 6: Method of Calculating Midline Between Two Similar Linear Fits of LC3-I and LC3-II for Unknown LC3 Sample

The two different lines 1 and n are linear fit equations of LC3-I and LC3-II, respectively. In order to find the midline of the two linear fits, an equation for calculating distances between the point P and the lines 1 and n is represented below.

The distances were calculated as follows.

$$\frac{\vert 2.8114 x - y - 1.0263 \vert}{\sqrt{{( 2.8114 )}^{2} + {( - 1 )}^{2}}} = \frac{\vert 2.8925 x - y - 1.0525 \vert}{\sqrt{{( 2.8925 )}^{2} + {( - 1 )}^{2}}} \qquad ( 2 )$$

Solving the above equation gives the following two different lines.

−0.3507x−y+0.004318=0 (3)

2.8514x−y−1.0392=0 (4)

Therefore, the equation for the midline m was as follows.

y=2.8514x−1.0392 (5)

LOD for the LC3 autophagy biomarker with a midline was calculated to be 63.61 M.

### Experimental Example 7: AuNR Synthesis and Surface Modification for LC3 Detection

Monodisperse AuNRs were synthesized using a binary surfactant mixture of CTAB and NaOL (Ye et al., 2013). Specifically, seeds were added to the growth solution, followed by culture overnight at 30° C. The clear AuNR solution turned red during the aging process and was then dispersed after centrifugation at 2200 g for 45 minutes. The CTAB molecules on the AuNR surface were replaced with functionalized PEG molecules. 40 mg of thiolated carboxyl PEG (Mw: 5,000) was stirred along with 2 mL of the AuNR solution (300 μg Au mL⁻¹) and incubated vigorously at 24° C. for 4 days. Unbound PEG molecules were then removed and the AuNR solution was dispersed after centrifugation at 2200 g for 4 minutes. The carboxyl group of PEG was activated and conjugated to the amine group of LC3-mAb by NHS/EDC reaction. 300 μL of the AuNR solution was incubated with 3 μL of 0.7 M EDC/NHS for 15 minutes, after which 150 μL of LC3-mAb (50 μg/mL) was added thereto. After centrifugation at 1000 g for 4 minutes, the supernatant was removed and the immuno-AuNR surface was prepared for LC3 detection.

### Experimental Example 8: Fabrication of Nanoplasmonic Platform for LC3 Detection

Cleanly washed microscope coverslip slides were pretreated with 10% (v/v) APTES for 1 hour, and then drop-coated with 10 μL of a diluted immuno-AuNR solution (OD: about 0.05). A closed water-bath microfluidic detection chamber including two coverslip slides with a gasket interposed therebetween was assembled and securely fixed for sample incubation and biosensing (FIG. 1). The detection chamber was then mounted on an automated dark-field microscope stage and a syringe pump was connected to the inlet of the detection chamber. Unbound immunogold on the surface of the coverslip slides was removed using ultrapure water at a flow rate of 100 μL min⁻¹ for 1 hour, followed by treatment with 1 mL of a 0.2 M ethanolamine solution for 30 minutes to inactivate unreacted functional groups. Thereafter, at least 10 μL of LC3 sample and PEBP1 (2 μg mL⁻¹) were injected into the chamber and incubated for 4 hours and 30 minutes, respectively, and contaminants were washed with ultrapure water before biosensing.

The LSPR wavelength of each immunogold was recorded three times throughout the experiment: 1) before LC3 sample injection, 2) after LC3 sample incubation, and 3) after PEBP1 treatment. The LSPR wavelength of each AuNR stabilized on the bottom coverslip slide was rapidly recorded with an imaging microscope system (SERA, NOST, Seoul, Korea). An automated surface scanning and imaging module with a high-resolution EMCCD detector (888, iXon Life, Oxford Instrument, Belfast, Northern Ireland) was connected to large-area (130×130 μm) computer-assisted high-speed LSPR mapping through a single click. The LSPR mapping results associated with the dark-field images were visualized on a monitor with RAON computer software (NOST, Seoul, Korea), and the plasmon shift (Δλmax) of each LSPR wavelength was calculated using OriginPro 9 software.

### Example 1: Preparation and Characterization of LC3 Nanoplasmonic Biosensor

Gold nanorods (AuNRs) were manufactured because exponential sensitivity thereof was high compared to gold nanospheres. Specifically, AuNRs were synthesized through a seed-mediated growth method using a binary surfactant CTAB and sodium oleate. The AuNRs thus synthesized were confirmed to have a monodisperse form with a size of 30.3 nm×112 nm and a rod shape with an aspect ratio of 3.69. Thereafter, CTAB molecules bound to the AuNR surface by electrostatic interaction were replaced with carboxymethyl-polyethylene glycol-thiol (CM-PEG-SH). This is because the thiol group of the PEG molecule has strong affinity for the gold surface through Au—S covalent bonding. Specifically, long-branched PEG stabilizes the AuNR surface by reducing steric hindrance and crosslinking reaction and preventing non-specific adsorption. Whether CTAB was successfully replaced with CM-PEG-SH was determined using UV-vis spectroscopy, zeta potential analysis, and X-ray photoelectron spectroscopy (XPS). Consequently, it was confirmed that the peak wavelength of the UV-vis absorbance spectrum was red-shifted by 3.0 nm because of the local RI change due to surface PEGylation. Also, based on results of zeta potential analysis of stability of AuNRs manufactured by replacing CTAB with CM-PEG-SH, AuNRs containing CTAB exhibited a high positive surface charge value of 49.94±4.21 mV due to specific characteristics of non-covalently bound CTAB. Through the PEGylation process, an Au—S covalent bond was formed between the thiol group of PEG and the AuNR surface, CTAB was replaced with the PEG moiety, and the surface of the nanoparticles was stabilized with a reduced zeta potential of 17.40±2.54 mV. Moreover, it was confirmed by XPS analysis that there was a rapid increase in O1s from 9.1% to 26.7%, indicating an increase in the carboxyl group at the end of the PEG branch (Table 1).

TABLE 1

| | % XPS elemental composition | | | | | |
|---|---|---|---|---|---|---|
| Sample | C₁ₛ | O₁ₛ | C₁₂ₚ | N₁ₛ | Br_{3d} | Si₂ₚ |
| CTAB-coated AuNR | 77.8 | 9.1 | — | — | — | 7.2 |
| PEGylated AuNR | 72.0 | 26.7 | 0.3 | — | 0.3 | — |
| Antibody Conjugated AuNR | 65.8 | 21.8 | 6.0 | 2.3 | — | — |

Thereby, it was confirmed that PEGylation replaced the CTAB molecule and made the surface accessible for antibody conjugation.

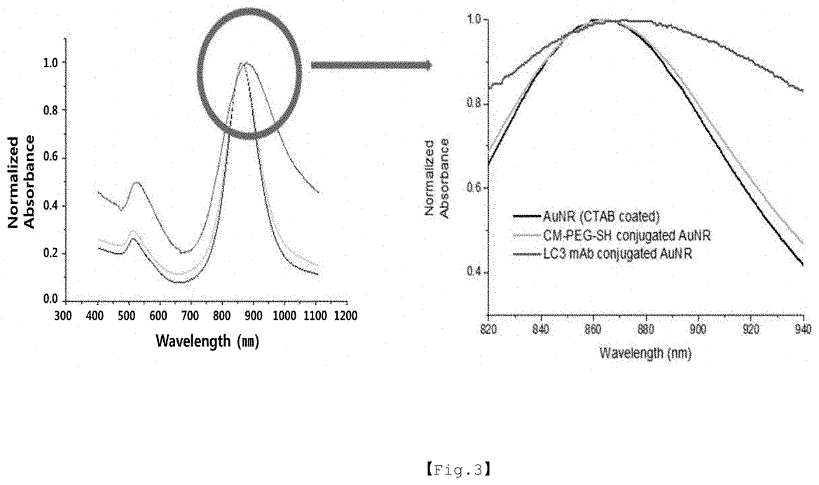

Following PEGylation, conjugation to a LC3 monoclonal antibody was performed by NHS/EDC reaction. The PEG carboxylic acid residue was activated to form an amide bond with the primary amine residue of the antibody. Based on results of determining whether LC3-mAb was conjugated, it was confirmed that the UV-vis absorbance peak wavelength was additionally red-shifted by 4 nm (FIG. 3).

In addition, it was confirmed by XPS analysis that LC3-mAb was successfully conjugated to the AuNR surface (Table 1). Based on results of elemental composition analysis through XPS, it was confirmed that the composition of the alkyl chain and amine residue was increased after conjugation of LC3-mAb, and specifically, C12p and N1 were increased by 5.7% and 2.3%, respectively. These results were similar to those conventionally known, confirming successful stabilization of LC3-mAb on the AuNR surface, which is a major factor for preparation of the LC3 nanoplasmonic biosensor.

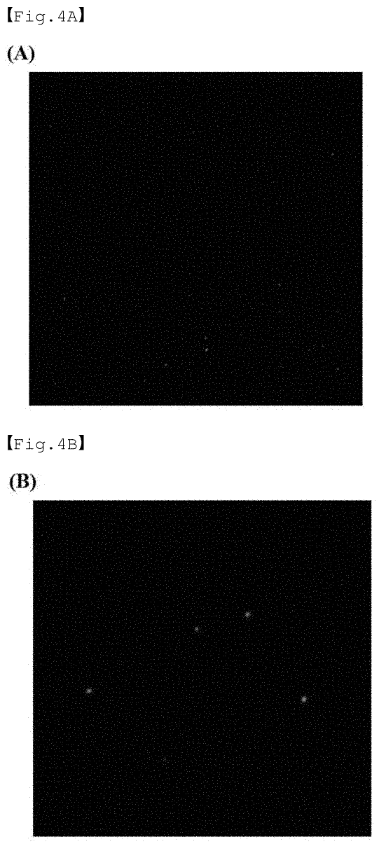

Then, the synthesized immuno-AuNRs were immobilized on 3-aminopropyl-triethoxysilane (APTES)-coated coverslip slides, and a closed detection chamber was assembled for preparation of an autophagy assay LSPR platform. Specifically, in order to eliminate the optical coupling effect of adjacent immuno-AuNRs and to utilize the platform as a single gold-nanoparticle-based LSPR sensor, red-orange AuNR particles were disposed at intervals 2.5 times larger than the nanoparticle diameter in the dark-field images (FIGS. 4A and 4B).

Each homogeneous nanoparticle distributed in a specific environment with the same local RI functions as an individual sensor providing reliable and reproducible Rayleigh light scattering signals. AuNRs are known to exhibit sensitive LSPR shifts due to dielectric constant changes around the nanoparticles. In particular, the longitudinal plasmon band (LPB) is very sensitive compared to the transverse plasmon band (TPB) (Truong et al., 2014). Therefore, the as-prepared single nanoparticle sensor generates a sensitive shift of the LPB band in response to the extended plasmon oscillation, and thus in this study, the LSPR peak shift was analyzed in the LPB band.

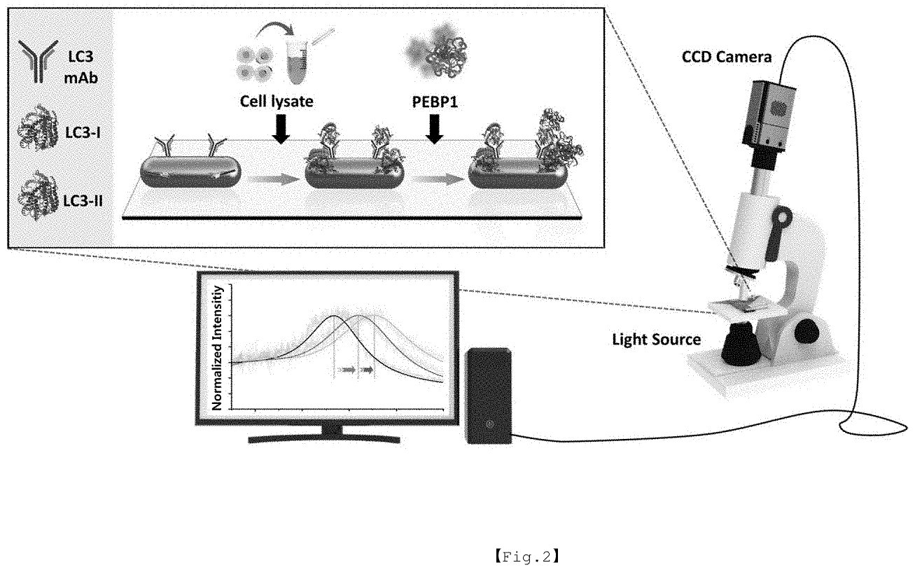

FIG. 2 schematically shows the entire process of the autophagy assay LSPR platform including two sequential LSPR sensing steps as the biosensor of the present invention. The first step is sensing both LC3-I and LC3-II using LC3-mAb on the immuno-AuNR surface, and the next second step is inducing an additional LSPR shift when only LC3-I adheres to PEBP1. The LC3 interacting region (LIR), composed of tryptophan (W) and leucine (L), is separated by two random amino acids (WXXL) therebetween and allows interaction with LC3. PEBP1 containing the LIR motif of WDGL (55-58 amino acids of human PEBP1) binds to LC3-I but not to LC3-II. In this respect, the LC3 nanoplasmonic biosensor of the present invention suggests that the total amount of LC3 and the specific composition of LC3-I and LC3-II may be analyzed within a single platform.

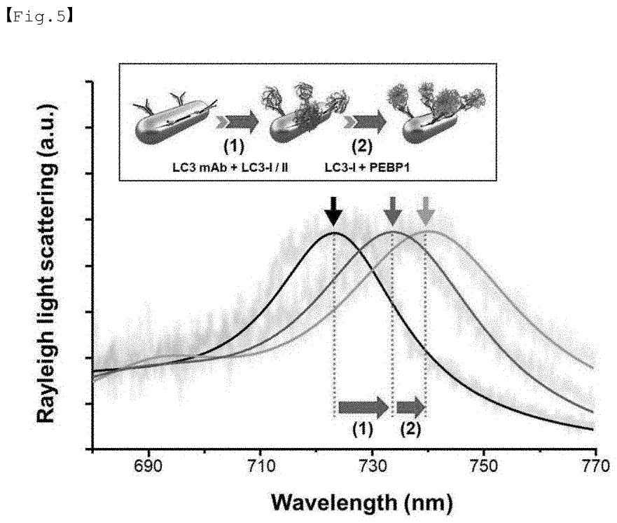

With reference to FIG. 5, a specific red-shift of the LPB for each LSPR sensing step can be confirmed. As shown in FIG. 5, an LSPR λmax red-shift of 8.90 nm was observed by the antibody-antigen reaction between LC3-mAb conjugated to the AuNR surface and LC3-I (50 μg mL⁻¹). Also, a Amax shift of 4.10 nm was additionally induced by injection of 2 μg mL⁻¹ PEBP1, which directly interacts with LC3-I captured by immuno-AuNRs. These results show that LC3 may be detected using the AuNR biosensor and also that each single particle functions as an individual sensor by inducing red-shift of the main surface plasmon resonance spectrum using changes in the refractive index and resonance characteristics around the nanoparticles.

### Example 2: Specificity and Sensitivity of Autophagy Assay Nanoplasmonic Biosensor

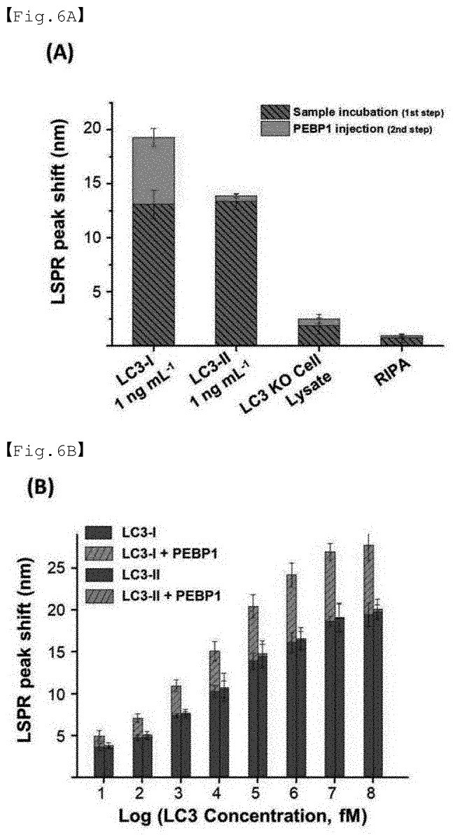

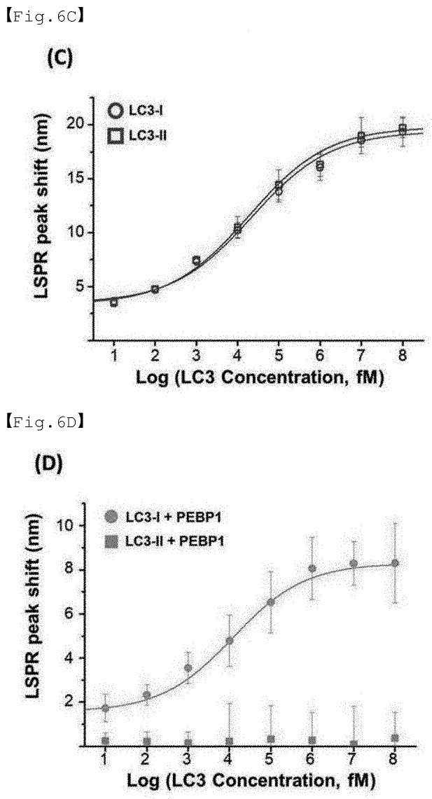

Human cells are known to consist of 2.7 million proteins per μm³, with an average of 6,000 different biomolecules. In this respect, cell lysates may cause non-specific reactions on the surface of immuno-AuNRs, reducing the sensitivity and reliability of LC3 detection. Therefore, non-specific adhesion was confirmed by incubation in LC3 knockout cell lysates compared to RIPA buffer and LC3 samples (FIG. 6A).

As the autophagy assay LSPR platform was processed in two consecutive signal measurement steps, LSPR shifts by sample and PEBP1 injection are shown in FIG. 6A in the form of a cumulative bar graph. As can be seen in FIG. 6A, in the first LSPR measurement step (gray boxes in FIG. 6A), the LC3 KO cell lysate and the RIPA buffer exhibited respective LSPR Δλmax values of 1.86±0.75 nm and 0.73±0.38 nm through non-specific absorption. Also, similar LSPR Δλmax values were measured for 1 ng mL⁻¹ of LC3-I and LC3-II (13.08±1.32 nm and 13.34±0.72 nm, respectively). These results suggest that the structures of LC3-I and LC3-II, which share the same LC3 backbone of four alpha helices and four beta sheets, are almost identical.

In addition, it was confirmed that only the LC3-I sample induced an LSPR peak shift when treated with PEBP1 (FIG. 6A). Specifically, after incubation with PEBP1 for 30 minutes, a further LSPR Δλmax of 6.20±0.82 nm was measured for the LC3-I sample, whereas for the LC3-II sample, LC3 KO cell lysate, and RIPA buffer input, only minor peak shifts were observed. Based on such results, non-specific binding of non-LC3 proteins was confirmed to be negligible, and there was an additional LSPR shift measurement only for LC3-I. Therefore, the novel nanoplasmonic biosensor of the present invention demonstrated selectivity for LC3 even in a situation where non-target proteins were prevalent in the sensing environment. In particular, it appeared that the two-step LSPR sensing platform could discriminate between LC3-I and LC3-II.

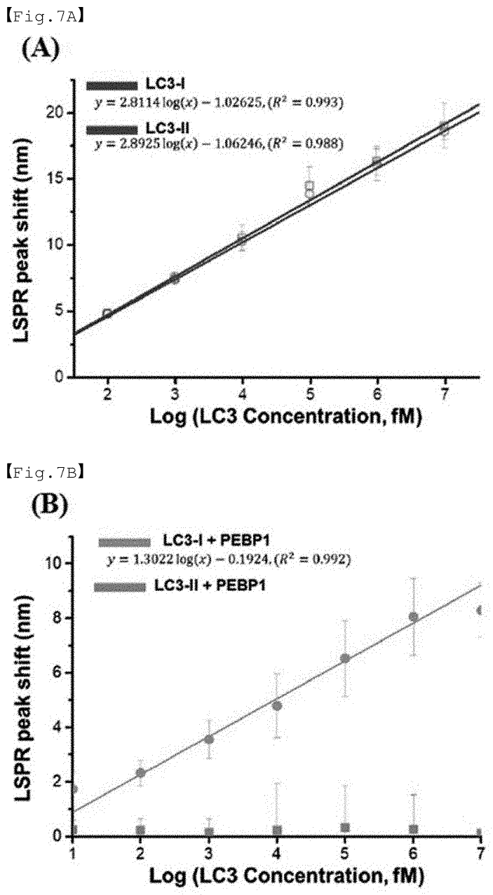

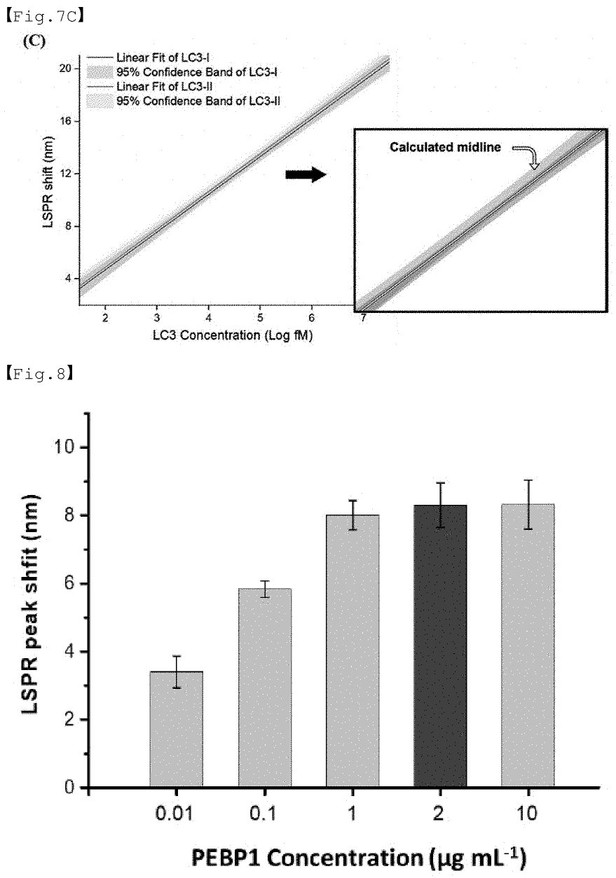

Next, using the two-step biosensor of the present invention, LC3-I and LC3-II samples were prepared in the concentration range of 10 fM to 100 nM and then the sensitivity of the two consecutive LSPR sensing processes was investigated. As can be seen in FIG. 6C, it was confirmed that LC3-II induced a slightly greater LSPR shift than LC3-I over the entire LC3 concentration range of the first LSPR sensing. This difference is deemed to be due to the phosphatidylethanolamine (PE) molecule conjugated to LC3-II. Specifically, charge density oscillation near the metal-dielectric surface is a major factor in LSPR biosensing, and considering that the sensing environment is water, PE is a small hydrophobic phospholipid with two fatty acid chains composed of 14 carbons weighing about 750 Da, and thus the hydrophobic PE molecule is expected to fold close to the LC3 surface without protruding outwards. Moreover, it is reported that a single PE molecule bound to the LC3 backbone does not affect the hexagonal lattice constant, the alkyl chain length of C14 in PE has a low dielectric constant of about 3.3 compared to a high dielectric charge of 20 to 30 on the protein surface, and the size of PE is also small relative to the consistent LC3 backbone (15 KDa). Therefore, considering these factors, it is expected that the differences in surface area and volume of LC3-I and LC3-II in the sensing environment are negligible, and the dielectric constant is not greatly different. Accordingly, for the first LSPR sensing, a larger but negligible LSPR shift of about 2.28% was confirmed in LC3-II compared to LC3-I. The LSPR Δλmax for both LC3-I and LC3-II increased steadily with the logarithm of LC3 concentration in the range of 10¹ fM to 10⁸ fM, but appeared to saturate at 10⁷ fM. Such results are attributed to the saturation of the LC3 antibody binding site on the immuno-AuNR surface when the LC3 concentration input is greater than 10⁷ fM. In particular, strong linearity of the LSPR shift was confirmed in the range of 10² to 10⁷ fM, and respective sensitivities for LC3-I and LC3-II were 0.993 and 0.988 and respective slopes thereof were 2.81 and 2.89, which were similar to each other (FIGS. 7A to 7C).

For an unknown LC3 sample, two similar LC3-I/II linear fits may be simplified to a single midline representing the entirety of LC3. The midline of two similar linear fits was present in the region where the 95% confidence intervals overlap (FIG. 7C), indicating that LC3 detection using the midline did not reveal significant differences. By calculating the linear function of the midline, the limit of detection (LOD=3*δ/slope: δ is the standard deviation of the blank sample and the slope is the slope of the calibration curve) was determined to be 63.61 fM (Song et al., 2020). Considering that the LOD of a commercial LC3 ELISA kit is about 10 ng L⁻¹, it can be found that the sensitivity confirmed using the biosensor of the present invention is 20-fold higher than that of current ELISA technology. Thus, these results showed that LC3 was sensed in the range of femtomolar concentration to nanomolar concentration regardless of the type thereof in the first LSPR sensing step.

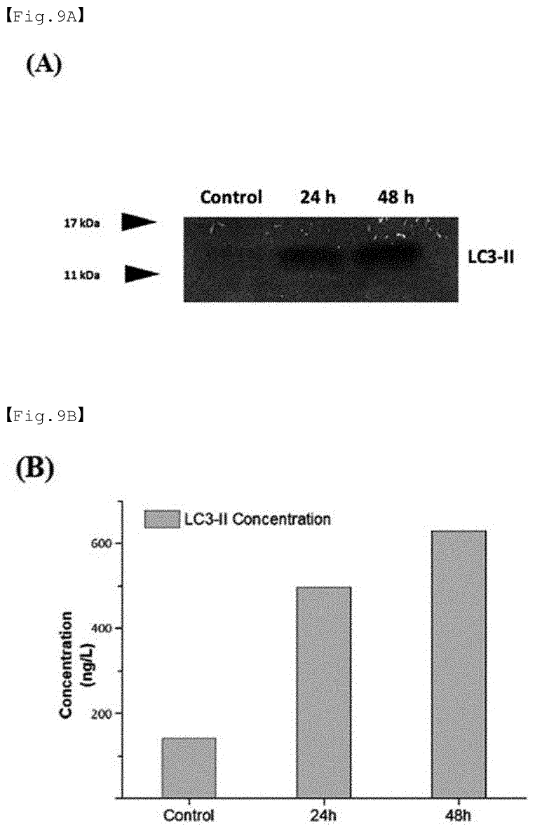

Next, specific LC3 forms, LC3-I or LC3-II, could be distinguished in the second LSPR sensing step. Specifically, in order to sensitively detect LC3-I in the second LSPR sensing step, PEBP1 was tested in a wide concentration range and the optimal concentration thereof was determined to be 2 μg mL⁻¹ (FIG. 8).

It was confirmed that only the LC3-I sample induced Δλmax in response to PEBP1 injection, and also that the LSPR shift was saturated after 10⁶ fM (FIG. 6D). Compared to the first LSPR sensing, the saturation point was lower when PEBP1 bound to LC3-I, which is deemed to be due to the relatively weak affinity of the LIR motif and the random orientation of the probe, resulting in lower affinity. PEBP1 (21 kDa), which is larger than LC3-I, reacts with the specific LIR binding region of LC3-I. However, when LC3-I binds to immune-AuNRs, the binding sites of LC3-I are randomly arranged, which may lead to conformational blockade for PEBP1 interaction and lower PEBP1 affinity.

Subsequent detailed analysis confirmed the strong linearity of the additional peak shift with respect to the logarithm of the concentration within the range of 10² to 10⁶ fM. Here, the linear regression equation was y=1.302 log(x)−0.192 (R²=0.992), indicating that the red-shift induced by PEBP1 was highly dependent on the LC3-I concentration (FIG. 7B). Based on linear regression, the LOD of PEBP1 reacting with LC3-I was calculated to be 16.88 fM, which is lower than the LOD of LC3 obtained in the first LSPR sensing step. The total LSPR shift with the addition of each Δλmax of two consecutive LSPR sensing steps was amplified only in the presence of LC3-I (FIG. 6A). These results indicate that the two-step LSPR platform of the present invention is capable of quantifying total LC3 with high sensitivity within a wide dynamic concentration range and distinguishing LC3-I thereamong.

### Example 3: Confirmation of Performance of Nanoplasmonic Biosensor of the Present Invention

Several methods of monitoring autophagic flux based on LC3 are currently being used, but are problematic in that they mainly depend on Western blotting, which has limitations in subjective interpretation, takes a lot of time, and lacks quantification. Hence, a fast and sensitive detection method that separately quantifies LC3-I and LC3-II is needed for accurate autophagic flux assay.

Lysates derived from cells and tissues in clinical applications are composed of LC3-I and LC3-II in various compositions and concentrations. Also, in order to analyze the effect of drugs on autophagy, it is important to accurately sense changes in the concentration and composition of LC3. In this respect, performance of the two-step nanoplasmonic biosensor of the present invention for quantifying LC3 was verified with LC3 samples of various LC3-I/II ratios before clinical application.

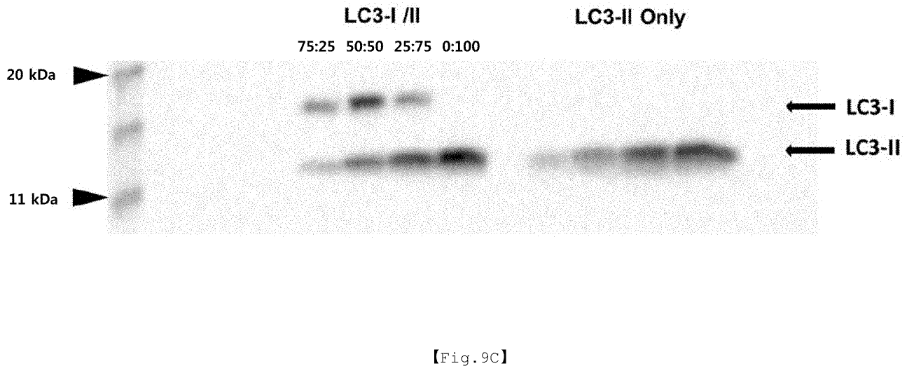

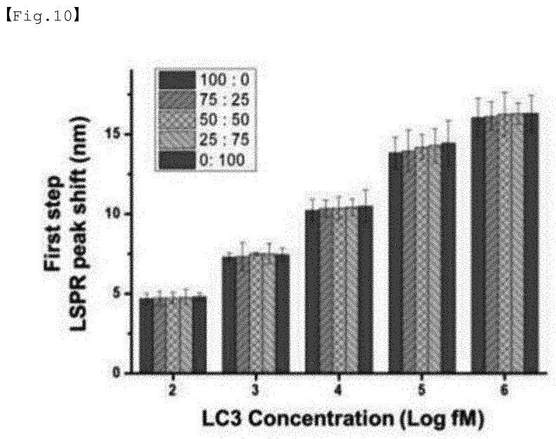

Specifically, pure LC3-I and LC3-II stocks were mixed to obtain samples of various LC3-I/II ratios with the same total LC3 concentration (FIGS. 9A to 9C).

In consideration of the LC3 concentrations in human clinical samples and cell lines, two consecutive LSPR peak shifts after LC3 were measured. Samples and PEBP1 were incubated separately.

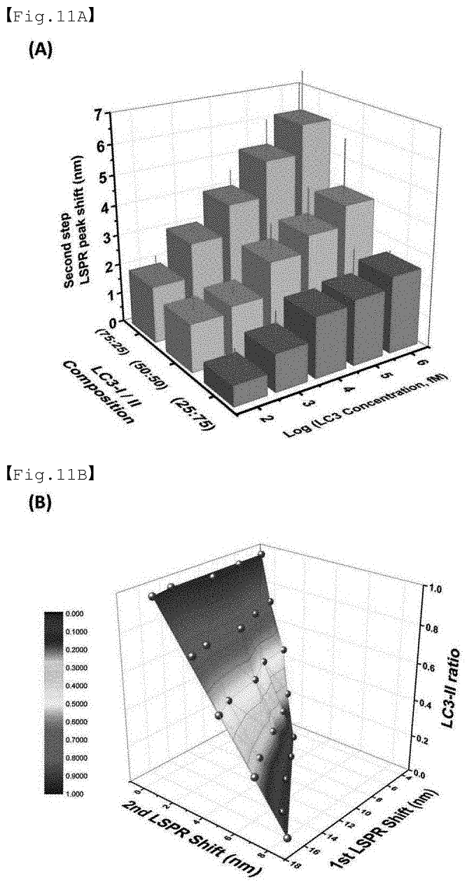

As can be seen in FIG. 10, similar to the results of FIG. 6C, it was confirmed that a difference in signal intensity of the first sensing step depending on the type of LC3 at each concentration was insignificant (FIG. 10).

These results show that the immuno-AuNR affinities for LC3-I and LC3-II are not different, which is deemed to be due to the use of a long-chain PEG molecule that serves as a linker connecting the AuNR surface to the antibody, thereby reducing steric hindrance of the binding site associated with the antibody. Therefore, Δλmax in the first LSPR sensing step mainly depends on the total LC3 concentration, which is similar to the experimental results of FIGS. 6A to 6D.

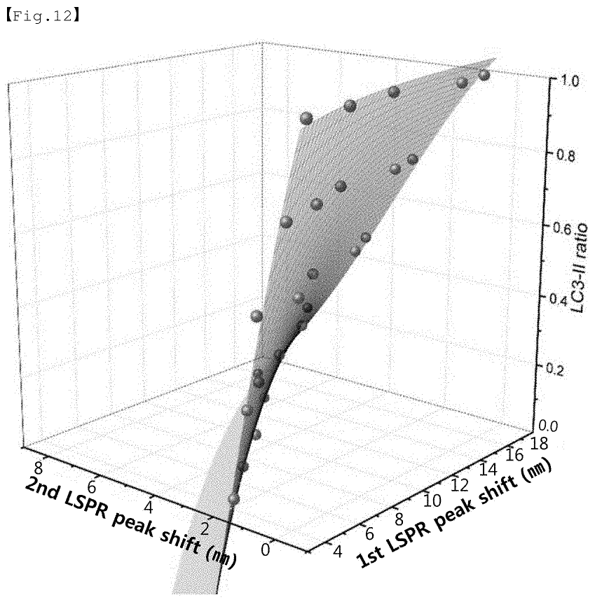

Unlike the first LSPR sensing step, a significant LSPR Δλmax was observed in the second sensing step. Within the same total LC3 concentration, the higher LC3-I composition induced a greater LSPR Δλmax (FIG. 11A). Also, similar to the detection result of unmixed LC3-I in FIGS. 6A to 6D, an additional LSPR peak shift showed a gradual increase in the reaction with PEBP1 in the concentration range of 10² to 10⁶ fM. These results suggest that the two-step LC3 detection platform of the present invention is capable of detecting total LC3 with high sensitivity and determining the LC3-I/II ratio based on two sequential LSPR sensing results. Thereafter, a total of 25 different experimental results (5×5 in the LC3 concentrations and compositions) were 3D plotted on the XYZ axes, and the two LSPR sensing results were represented in relation to the LC3-I/II ratio (FIG. 11B). Here, the LC3-II ratio out of total LC3 was defined as Z, and two sequential Δλmax measurements were defined as X and Y, respectively. As can be seen in FIG. 11B, a distorted surface filled with a red portion representing a higher LC3-II composition was observed by connecting 25 dots with adjacent dots. In addition, a more accurate correlation between X, Y, and Z was investigated using computer software (OriginPro 9) to obtain Equation 1 with a high correlation (R²=0.9795) (FIG. 12). Therefore, it is expected that the LC3 ratio in the unknown sample can be determined by substituting the two measured values obtained using the LC3 nanoplasmonic biosensor of the present invention into the surface equation (Equation 1).

Therefore, the two-step LSPR biosensor of the present invention was proven to be reliable in complex sample analysis similar to clinical conditions, and enabled autophagic flux assay by quantifying LC3-I and LC3-II within the concentration range of 10² to 10⁶ fM.

### Example 4. Potential of Nanoplasmonic Biosensor for Screening Autophagy-Targeting Drug

In order to determine whether the nanoplasmonic biosensor of the present invention is able to be useful as a tool for screening an autophagy-targeting drug, two different experimental conditions were prepared and the LC3 expression level was analyzed for each case. A Caov-3 (human ovarian cancer cell) cell line was treated with bafilomycin A (Baf A) well-known as an autophagy inhibitor, and HER2 positive and negative breast cancer cell lines were treated with trastuzumab commonly used in human epidermal growth factor receptor 2 (HER2) overexpressing breast cancer chemotherapy.

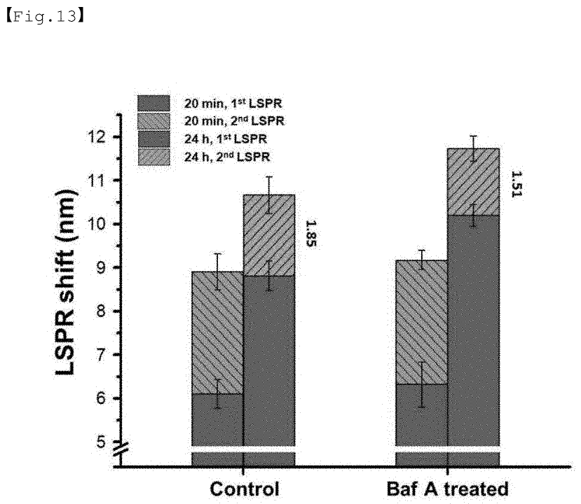

Then, the LSPR peak shift of the Caov-3 cell line treated with bafilomycin A or not treated therewith was represented in the form of a cumulative bar graph, as shown in FIG. 13. Here, the regular and hatched boxes represent the measured first and second LSPR shifts, respectively, and error bars correspond to the standard errors measured in 60 immuno-AuNRs.

Bafilomycin A (Baf A) inhibits autophagosome-lysosome fusion and autolysosome acidification. Thus, autophagic vacuoles accumulate and autophagosome contents are not degraded, resulting in accumulation of LC3-II over time and thus an increase in total LC3 levels (Mauvezin and Neufeld, 2015). The Caov-3 cell line, which showed a good response to autophagy inhibitors in LC3 expression pattern changes, was treated with Baf A for 20 minutes and 24 hours to mimic clinical chemotherapy conditions. Thereafter, the effect of Baf A targeting autophagy activity was confirmed by comparing the two samples in the presence and absence of Baf A (FIGS. 13 and 14). As can be seen in FIGS. 13 and 14, the total concentration of LC3 was increased over time, and LC3-II accumulation was induced in Baf A-treated cells (FIG. 14).

In addition, substituting the two LSPR peak shifts in the samples treated with Baf A for 20 minutes (red cumulative bar graphs in FIG. 13) into the surface equation (Equation 1 below) resulted in low LC3-II compositions of 5.26% and 6.78%, respectively.

Z=1−2.072×(Y−0.3265)/(X−0.3974) [Equation 1]

Here, X is the first LSPR peak shift value, Y is the second LSPR peak shift value, Z is the LC3-II ratio, and R²=0.9805.

These low LC3-II composition and concentration values are similar to the results reported in previous studies (Guo et al., 2013) and are also similar to the results of Western blotting. In addition, when comparing the Baf A-treated samples with the control group, a larger LSPR shift in the first sensing step (a blue box in FIG. 13) and a smaller LSPR shift in the second sensing step (a light blue hatched box in FIG. 13) appeared. This indicates that the total LC3 concentration was increased and the LC3-I composition was decreased upon treatment with Baf A. The two consecutive LSPR peak shifts of the LC3-II composition were 8.81±0.34 nm and 1.85±0.42 nm for the 24-hour control sample and were 10.2±0.25 nm and 1.53±0.29 nm for the 24-hour Baf A-treated sample, and calculated to be 65.5% and 78.8%. Plasmon results were also demonstrated by a thicker LC3-II band for Baf A-treated cells in Western blotting (FIG. 14). This is deemed to be because the total LC3 concentration and LC3-II accumulation increased due to inhibition of autophagosome degradation.

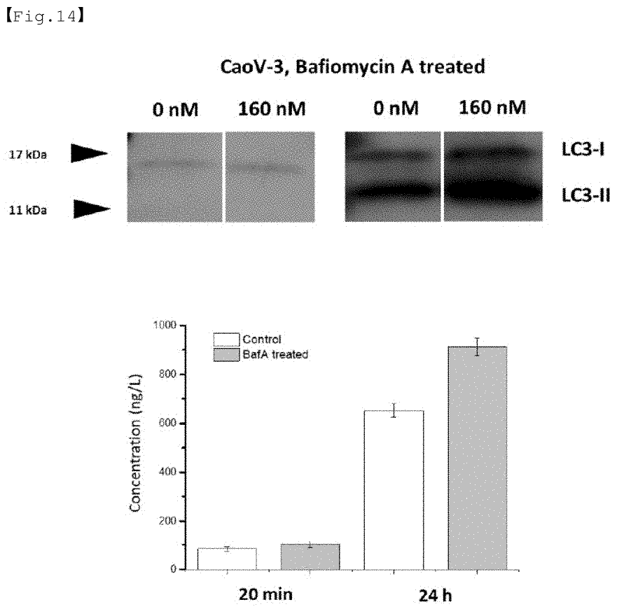

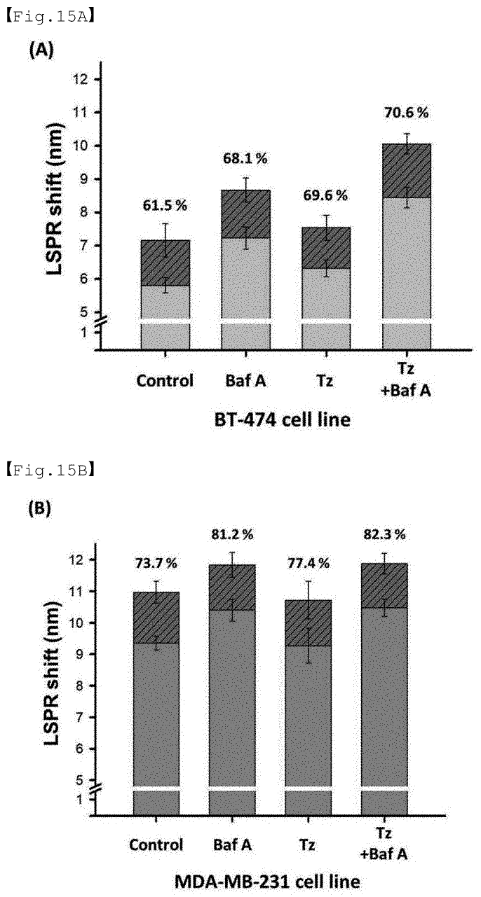

The LSPR peak shifts of cell lysates derived from two different human breast cancer cell lines under different conditions (20 μg mL⁻¹ trastuzumab and 50 nM bafilomycin A for 12 hours) were measured, and the results thereof are shown in FIGS. 15A and 15B. Here, the regular and hatched boxes are the measured first and second LSPR shifts, respectively, and the percentage above each bar graph is the LC3-II ratio calculated by substituting the experimental results into Equation 1. Also, error bars are standard errors measured in 60 immuno-AuNRs.

In addition, trastuzumab (Tz, Herceptin), which is another well-known anticancer drug, was measured in two different breast cancer cell lines, BT-474 and MDA-MB-231, in order to determine whether Tz therapy involved autophagy signaling. Trastuzumab is a humanized monoclonal antibody that specifically binds to HER2, which is overexpressed in about 20% of human breast cancer and is associated with poor prognosis (Press et al., 1997). Thus, since HER2 is overexpressed in BT-474 but underexpressed in MDA-MB-231, responses to Tz treatment are expected to be different, and measuring the LC3 expression level therebetween will reveal whether Tz chemotherapy involves the autophagic pathway.

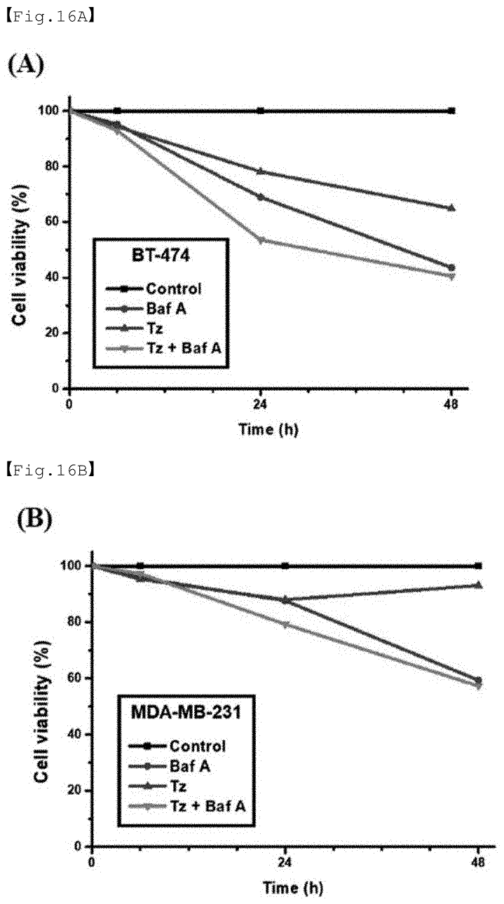

As can be seen in FIGS. 16A and 16B, when treated with Baf A (50 nM) and Tz (20 μg mL⁻¹), both cells showed gradual cell death with Baf A treatment over time, but when treated with Tz, only the number of BT-474 cells gradually decreased (FIGS. 16A and 16B).

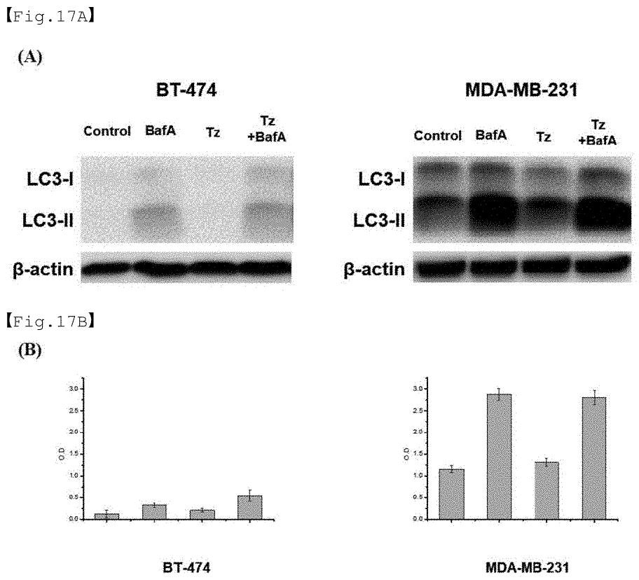

These results are consistent with previous studies showing that Baf A induces cell death and that Tz therapy is only effective for BT-474 (HER2-overexpressing), rather than MDA-MB-231 (HER2-underexpressing). Therefore, it was confirmed that cancer cells were correctly treated with each drug, and cell lysates were prepared from 12 hours and used for detailed LC3 expression analysis (FIGS. 15A and 15B and 17A and 17B).

When comparing the LSPR peak shifts of the cell lines in FIGS. 15A and 15B, the entire BT-474-derived sample showed a smaller Δλmax in the first sensing step, resulting in a lower LC3 expression level in BT-474, which indicates low basal autophagy activity. Similar results were confirmed in Western blotting and ELISA (FIGS. 17A and 17B), indicating that HER2-amplified tumors showed low LC3 expression levels. In both cell lines, Baf A induced a greater first-step LSPR Δλmax and an increased LC3-II ratio (percentages above the bar graphs in FIGS. 15A and 15B) due to LC3 accumulation as previously observed. However, considering that autophagy measurement should be interpreted depending on treatment with or without an inhibitor (Baf A in this case), only the BT-474 cell line showed a significant first-step LSPR Δλmax increase depending on Tz treatment. The first-step LSPR Δλmax was increased from 7.23±0.33 nm to 8.44±0.31 nm and the LC3-II ratio was slightly increased from 68.1% to 70.6% due to additional treatment with Baf A in Tz (FIG. 15A). For MDA-MB-231, a negligible Δλmax (from 10.40±0.35 nm to 10.48±0.28 nm) was measured. These results suggest that Tz treatment for HER2 positive breast cancer cells increased total LC3 concentration and LC3-II conversion, consistent with previous studies. This increased autophagic flux with trastuzumab treatment may be deemed to be because a decrease in endogenous HER2 inhibits HER2 binding to the essential autophagy protein Beclin 1 and consequently induces autophagic flux.

Therefore, it was confirmed that the LC3 assay nanoplasmonic biosensor of the present invention was able to sense changes in the LC3 expression level of all cancer cell lines tested after Baf A treatment, and also that trastuzumab enhanced the autophagic flux only for the HER2 positive breast cancer cell line. In case of LC3 Western blotting, there was no significant difference in LC3 expression level due to low sensitivity (FIGS. 17A and 17B), whereas the two-step LSPR-based autophagy assay platform using the biosensor of the present invention has potential as a rapid and sensitive drug screening tool in the field of autophagy-targeting cancer therapy.

In conclusion, the novel plasmonic nanoparticle-based two-step LSPR sensing platform capable of accurate LC3 turnover calculation for autophagic flux assay according to the present invention is capable of exhibiting 20-fold higher sensitivity than conventional LC3 ELISA by quantifying total LC3 in the range of femtomolar concentration to nanomolar concentration, and of quantifying individual LC3 forms by measuring the LC3 composition when compared to relative values obtained through Western blotting. Moreover, experiments using samples mimicking clinical samples in which LC3-I and LC3-II are mixed show that the biosensor of the present invention provides reliable and accurate sensing results and a specific equation representing the correlation between LSPR shift and LC3 composition is obtained. In addition, the LC3 expression levels in drug-treated human cancer cell lines can be classified in detail, and the efficacy of autophagy-targeting drugs can be determined using the new nanoplasmonic biosensor. Ultimately, the biosensor of the present invention suggests clinical applicability due to rapid sensing, cost-effectiveness, and high sensitivity of the LSPR platform. Furthermore, the LC3 nanoplasmonic biosensor can be utilized not only for new drug development but also for research into the overall relationship between autophagy and cancer, such as cancer diagnosis, treatment, prognosis, and the like.

As is apparent from the above description, a nanoplasmonic biosensor according to the present invention is capable of detecting LC3 as an autophagy marker using the plasmon resonance effect without an additional marker, particularly detecting the same with low sensitivity and in a wide concentration range, and moreover, quantifying the ratio of LC-I to LC3-II to effectively measure an autophagy marker based thereon. Therefore, the nanoplasmonic biosensor according to the present invention can be variously utilized in detection of complex mixtures, early detection of cancer, screening of cancer therapeutic agents, and the like.

From the above description, those skilled in the art to which the present invention belongs will understand that the present invention may be embodied in other specific forms without changing the technical spirit or essential characteristics thereof. In this regard, the embodiments described above should be understood to be non-limiting and illustrative in every way. The scope of the present invention should be construed as including, rather than the above detailed description, all changes or modifications derived from the meaning and scope of the following claims and equivalents thereto.

What is claimed is:

1. A method of detecting an autophagy marker with a nanoplasmonic biosensor for measuring an autophagy marker with high sensitivity comprising a substrate, immunogold nanorods immobilized on the substrate and linked with a monoclonal antibody specifically binding to an autophagy marker, and a measurement unit configured to measure localized surface plasmon resonance in the immunogold nanorods, the method comprising:
(1) measuring a Rayleigh scattering spectrum and obtaining a maximum wavelength shift therefrom by bringing a biomarker mixture into contact with immunogold nanorods to induce specific binding to a monoclonal antibody; and
(2) measuring a Rayleigh scattering spectrum and obtaining a maximum wavelength shift therefrom by inducing specific binding to LC3-I after PEBP1 injection

2. The method according to claim 1, wherein the immunogold nanorods have an aspect ratio of 3 to 4.

3. The method according to claim 1, wherein the biosensor detects the autophagy marker by measuring a change in Rayleigh scattering spectrum generated by specific binding of the autophagy marker.

4. The method according to claim 1, wherein the autophagy marker is LC3.

5. The method according to claim 4, wherein the LC3 comprises LC3-I and LC3-II.

6. The method according to claim 1, wherein the monoclonal antibody is LC3-mAb.

7. The method according to claim 1, wherein the biosensor detects the autophagy marker in a range of femtomolar concentration (fM) to nanomolar concentration (nM).

8. The method according to claim 1, wherein the biosensor detects the autophagy marker at a low limit of detection ranging from 10² fM to 10⁶ fM.

9. The method according to claim 1, further comprising treating a surface of the substrate of the biosensor, on which the immunogold nanorods are immobilized, with carboxymethyl-polyethylene glycol-thiol.

10. The method according to claim 1, wherein the biomarker mixture is a cancer-cell-derived lysate.

11. A method of determining autophagic flux with a nanoplasmonic biosensor for measuring an autophagy marker with high sensitivity comprising a substrate, immunogold nanorods immobilized on the substrate and linked with a monoclonal antibody specifically binding to an autophagy marker, and a measurement unit configured to measure localized surface plasmon resonance in the immunogold nanorods, the method comprising:
(1) measuring a Rayleigh scattering spectrum and obtaining a maximum wavelength shift therefrom by bringing a biomarker mixture into contact with immunogold nanorods to induce specific binding to a monoclonal antibody; and
(2) measuring a Rayleigh scattering spectrum and obtaining a maximum wavelength shift therefrom by inducing specific binding to LC3-I after PEBP1 injection.

12. The method according to claim 11, wherein the method quantifies a total concentration of LC3 in a sample in which LC3-I and LC3-II are mixed at various concentrations, and simultaneously quantifies a ratio of LC3-I to LC3-II.

13. The method according to claim 11, further comprising (3) measuring a ratio of LC3-I to LC3-II by substituting the two maximum wavelength shift values obtained in steps (1) and (2) into Equation 1 below:
Z=1−2.072×(Y−0.3265)/(X−0.3974) [Equation 1]
wherein X is a first LSPR peak shift value, Y is a second LSPR peak shift value, Z is a LC3-II ratio, and R²=0.9805.

14. The method according to claim 11, wherein the autophagic flux is analyzed by quantifying LC3-I and LC3-II within a concentration range of 10² to 10⁶ fM.

15. A method of screening a cancer-targeting drug candidate with a nanoplasmonic biosensor for measuring an autophagy marker with high sensitivity comprising a substrate, immunogold nanorods immobilized on the substrate and linked with a monoclonal antibody specifically binding to an autophagy marker, and a measurement unit configured to measure localized surface plasmon resonance in the immunogold nanorods, the method comprising:
(1) treating a cancer cell line with an autophagy inhibitor;
(2) measuring a first maximum wavelength shift value by bringing a cell lysate isolated from the cancer cell line after step (1) into contact with immunogold nanorods, and measuring a second maximum wavelength shift value after PEBP1 injection;
(3) treating the cancer cell line with a cancer-targeting drug candidate;
(4) measuring a first maximum wavelength shift value by bringing a cell lysate isolated from the cancer cell line after step (3) into contact with immunogold nanorods, and measuring a second maximum wavelength shift value after PEBP1 injection;
(5) calculating a composition of LC3-I and LC3-II by substituting the first and second maximum wavelength shift values obtained in steps (2) and (4) into Equation 1 below; and
(6) determining the drug candidate to be a cancer therapeutic agent when LC3 and LC3-II values calculated in step (4) are increased compared to LC3 and LC3-II values calculated in step (2):
Z=1−2.072×(Y−0.3265)/(X−0.3974) [Equation 1]
wherein X is a first LSPR peak shift value, Y is a second LSPR peak shift value, Z is a LC3-II ratio, and R²=0.9805.

16. The method according to claim 15, wherein the autophagy inhibitor comprises bafilomycin A, chloroquine, 3-methyladenine, or combinations thereof.

17. The method according to claim 15, wherein the cell lysate comprises LC3-I and LC3-II that are mixed at various concentrations.
